(19) United States
(12) Patent Application Publication
(10) Pub. No.: US 2026/0299168 A1
(43) Pub. Date: Oct. 1, 2026

(54) Contact Lenses With Collagen Fragments

(71) Applicant: Bausch + Lomb Ireland Limited, Dublin (IE)

(72) Inventors: Jade J. Russell, Perry, NY (US); Alok K. Awasthi, Pittsford, NY (US); James Anthony DiBella, Penfield, NY (US)

(73) Assignee: Bausch + Lomb Ireland Limited, Dublin (IE)

(21) Appl. No.: 19/570,324

(22) Filed: Mar. 18, 2026

Related U.S. Application Data
(60) Provisional application No. 63/774,463, filed on Mar. 19, 2025.

(51) Int. Cl. G02B 1/04; C07K 14/78

(52) U.S. Cl. CPC G02B 1/043; C07K 14/78

(57) ABSTRACT

The present invention relates to ophthalmic devices, in particular contact lenses, which are capable of gradually releasing one or more collagen fragments during wear. The present invention also provides methods for making ophthalmic devices of the invention and for using time-controlled release of one or more collagen fragments for improving comfort and/or treating eye conditions, such as dry eye disease.

### CROSS-REFERENCE TO RELATED APPLICATIONS

The present application claims the benefit of priority to U.S. Provisional Patent Application No. 63/774,463, filed Mar. 19, 2025, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 16, 2026, is named 01426-0008-00US and is 3,924 bytes in size.

### FIELD

The present invention relates to ophthalmic devices, in particular contact lenses, which are capable of gradually releasing one or more collagen fragments during wear. The present invention also provides methods for making ophthalmic devices of the invention and for using time-controlled release of one or more collagen fragments for improving comfort and/or treating eye conditions, such as dry eye disease.

### BACKGROUND

It is highly desirable that contact lens be as comfortable as possible for wearers. There is a decades long need to improve the comfort of the lenses. While many attempts have been made to address this problem, an estimated 50% of people who wear contact lenses still experience dryness or eye irritation throughout the day and particularly towards the end of the day. In fact, contact lens discomfort is recognized as the most common cause of contact lens wearer drop-out over the past four decades.

Furthermore, Keratoconjunctivitis sicca, also known as dry eye disease (DED), is a multifunctional disorder of the tear film, and ocular surface which results in discomfort, visual disturbance, and often even in ocular surface damage. The two mechanisms that are being discussed as pivotal in the etiology of this disease and which also appear to reinforce each other mutually are tear hyperosmolarity and tear film instability. Hyperosmolar tear fluid can result from excessive tear film evaporation or reduced aqueous flow. It activates an inflammatory cascade and causes the release of inflammatory mediators into the tear fluid, with multiple pathophysiological effects eventually leading to further increased tear film evaporation and tear film instability. Thus, tear film instability can be a consequence of hyperosmolarity. Alternatively, tear film instability can also develop through its own etiological pathway, for example via abnormalities of the lipid layer composition, such as from meibomian gland disease.

There are a variety of factors that are involved in the creation, propagation, and exacerbation of DED symptoms in the contact lens wearing population, including lens materials, thickness, water content, coatings, and care solutions. These variables can be controlled to some extent by the clinician, who may try alternatives to improve patient comfort and compliance. Other factors which are not so easily altered are those which affect the tear film such as environment and physiological variety.

The tear film consists of a delicate balance between aqueous, mucin, and lipid constituents. It is suspected that the presence of a contact lens causes instability in the tear film which can lead to discomfort and reduction in wear time. It has been suggested that this is due to the lens causing lipid and mucin abnormalities, increasing evaporation which subsequently causes symptoms of dryness.

One of the widely used approaches to improve ocular comfort with contact lenses is to apply directly eye drops of an ocular lubricant into the wearer's eye while the lens is being worn, in order to provide some relief to some extent, e.g., the initial discomfort of wearers, discomfort suffering from dry-eye effects, or end-of-day discomfort. However, there are unavoidable disadvantages with this approach. For example, eye drops are typically applied only after a lens wearer is already suffering discomfort and as such do not prevent the discomfort from occurring. Furthermore, a user needs to easily and conveniently access eye drops to ease the discomfort and therefore must carry a bottle of eye drops with him/her. This adds cost and inconvenience to the lens wearers. Even more so, the high viscosity of such drops can lead to transient blurriness and/or reduced light transmissivity upon installation, leading to reduced vision and even safety concerns.

Recently, surfactants, lubricants or other additives are added in the lens packaging solution to ease to some extent initial discomfort and other symptoms (see, for example, U.S. Pat. Nos. 5,882,687, 5,942,558, 6,348,507, 6,440,366, 6,531,432, and 6,699,435; and Published PCT Patent Applications WO9720019 and WO2006/088758). However, although such approach may alleviate, to some extent, some forms but not all forms of discomfort, especially the end-of-day comfort, dry-eye symptoms and/or contact lens induced dry-eye symptoms.

In addition, leachable lubricants are incorporated in lens formulations for making contact lenses to alleviate some discomfort symptoms (see for example, U.S. Pat. Nos. 6,822,016 and 6,367,929, U.S. Patent Application Publication No. US 2006/0251696 A1). Although the methods disclosed in the above patents and patent application may alleviate some discomfort symptoms to some extent, not all symptoms can be addressed and/or reduced.

Therefore, there exists a need for hydrogel soft contact lenses which not only have initial insertion comfort but also are comfortable during the period of wear.

### SUMMARY OF THE INVENTION

The present invention relates to ophthalmic devices, in particular contact lenses, which gradually release one or more collagen fragments during wear. The present invention also provides methods for making ophthalmic devices of the invention and for using time-controlled release of one or more collagen fragments for improving comfort and/or treating eye conditions, such as dry eye disease.

The present invention, in one aspect, provides a soft hydrogel contact lens, comprising a one or more collagen fragments, wherein the collagen fragments are not covalently linked to the contact lens.

The present invention, in another aspect, provides a soft hydrogel contact lens, comprising a polymer matrix and one or more collagen fragments which are not covalently linked to the polymer matrix. Such collagen fragments may be fully within the lens, but preferably, at least some of the collagen fragments have at least a portion of their structure at the surface of the soft hydrogel lens. Such soft contact lenses are preferably capable of gradually releasing the collagen fragment over an extended period of wearing time.

In one aspect of the invention, the soft contact lenses comprising one or more collagen fragments not covalently bound to the lens exhibit at least one characteristic selected from the group consisting of the following:
a WBUT of at least about 10 seconds;
a sessile drop water contact angle of about 80 degrees;
a mean static COF of less than 0.4;
a mean low kinetic COF of less than 0.25; and
a mean high kinetic COF of less than 0.4.

In yet another aspect of the invention, the invention provides a soft contact lens comprising a lens body and a collagen fragment not covalently bound to the lens body; wherein the lens body is formed from a contact lens material selected from the group consisting of etafilcon, hilafilcon A, hilafilcon B, nelfilcon, nesofilcon, ocufilcon, omafilcon, aquafilcon, balafilcon, comfilcon, enfilcon, galyfilcon, kalafilcon, lenefilcon, lotrafilcon A, lotrafilcon B, samfilcon, and senofilcon.

The present invention, in another aspect, provides a process for making a soft contact lens comprising the steps of: a) obtaining an uncured lens forming composition comprising one or more collagen fragments; b) introducing an amount of the uncured lens forming composition in a mold for making a contact lens; c) curing the uncured lens forming composition in the mold to form a soft contact lens with the collagen fragment being not covalently linked to the polymer matrix but being distributed therein in a substantially uniform manner; d) packaging the resultant soft contact lens in a container containing a packaging solution; and e) sterilizing the soft contact lens in the package.

In yet another aspect of the invention a process is provided for making a soft contact lens capable of gradually releasing a collagen fragment over an extended period of wearing time comprising the steps of: a) obtaining an uncured lens forming composition; b) introducing an amount of the uncured lens forming composition in a mold for making a contact lens; c) curing the uncured lens forming composition in the mold to form a soft contact lens; d) swelling the resulting soft contact lens with a composition comprising one or more collagen fragments causing said collagen fragments to enter the polymer matrix and be distributed therein; e) packaging the resultant soft contact lens in a container containing a packaging solution; and f) sterilizing the soft contact lens in the package.

The present invention, in yet another aspect, provides an ophthalmic product comprising a sealed package which include a packaging solution and a soft hydrogel contact lens, wherein the hydrogel contact lens comprises a polymer matrix and a collagen fragment which is not covalently linked to the polymer matrix but distributed therein. Such soft contact lenses are preferably capable of gradually releasing the collagen fragment over an extended period of wearing time.

These and other aspects of the invention will become apparent from the following description of the presently preferred embodiments. The detailed description is merely illustrative of the invention and does not limit the scope of the invention, which is defined by the appended claims and equivalents thereof. As would be obvious to one skilled in the art, many variations and modifications of the invention may be affected without departing from the spirit and scope of the novel concepts of the disclosure.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings.

An “uncured lens forming composition” refers to a composition including reactive ingredients, and optionally non-reactive ingredients, used in forming contact lenses. The ingredients can include one or more hydrophilic monomers, oligomers, macromers, or polymers; and/or one or more hydrophobic monomers, oligomers, macromers, or polymers; and/or one or more silicone-containing monomers, oligomers, macromers, or polymers; and/or one or more crosslinkable prepolymers; or any combinations thereof. The uncured lens forming composition is capable of being crosslinked and/or polymerized thermally or actinically to form a hydrogel polymer matrix of a soft contact lens;

A “hydrogel” refers to a polymeric material which can absorb at least 10 percent by weight of water when it is fully hydrated. A hydrogel material can be obtained by polymerization or copolymerization of at least one hydrophilic monomer in the presence of or in the absence of additional monomers and/or macromers or by crosslinking of a prepolymer.

A “silicone hydrogel” refers to a hydrogel obtained by copolymerization of a polymerizable composition comprising at least one silicone-containing vinylic monomer or at least one silicone-containing macromer or a silicone-containing prepolymer.

“Hydrophilic,” as used herein, describes a material or portion thereof that will more readily associate with water than with lipids.

A “monomer” means a low molecular weight compound that can be polymerized actinically or thermally or chemically. Low molecular weight typically means average molecular weights less than 700 Daltons.

As used herein, “actinically” in reference to curing or polymerizing of a polymerizable composition or material or a lens-forming material means that the curing (e.g., crosslinked and/or polymerized) is performed by actinic irradiation, such as, for example, UV irradiation, ionized radiation (e.g. gamma ray or X-ray irradiation), microwave irradiation, and the like. Thermal curing or actinic curing methods are well-known to a person skilled in the art. Lens-forming materials are well known to a person skilled in the art.

A “vinylic monomer”, as used herein, refers to a low molecular weight compound that has an ethylenically unsaturated group and can be polymerized actinically or thermally. Low molecular weight typically means average molecular weights less than 700 Daltons.

The term “ethylenically unsaturated group” or “olefinically unsaturated group” is employed herein in a broad sense and is intended to encompass any groups containing at least one >C═C< group. Exemplary ethylenically unsaturated groups include without limitation acryloyl, methacryloyl, allyl, vinyl, styrenyl, or other C═C containing groups.

A “hydrophilic vinylic monomer”, as used herein, refers to a vinylic monomer which is capable of forming a homopolymer that can absorb at least 10 percent by weight water when fully hydrated. Suitable hydrophilic monomers are, without this being an exhaustive list, hydroxyl-substituted lower alkyl (C₁ to C₈) acrylates and methacrylates, acrylamide, methacrylamide, (lower allyl)acrylamides and -methacrylamides, ethoxylated acrylates and methacrylates, hydroxyl-substituted (lower alkyl)acrylamides and -methacrylamides, hydroxyl-substituted lower alkyl vinyl ethers, sodium vinylsulfonate, sodium styrenesulfonate, 2-acrylamido-2-methylpropanesulfonic acid, N-vinylpyrrole, N-vinyl-2-pyrrolidone, 2-vinyloxazoline, 2-vinyl-4,4′-dialkyloxazolin-5-one, 2- and 4-vinylpyridine, vinylically unsaturated carboxylic acids having a total of 3 to 5 carbon atoms, amino(lower alkyl)-(where the term “amino” also includes quaternary ammonium), mono(lower alkylamino)(lower alkyl) and di(lower alkylamino)(lower alkyl)acrylates and methacrylates, allyl alcohol and the like.

A “macromer” refers to a medium to high molecular weight compound or polymer that contains functional groups capable of undergoing further polymerizing/crosslinking reactions. Medium and high molecular weight typically means average molecular weights greater than 700 Daltons. Preferably, a macromer contains ethylenically unsaturated groups and can be polymerized actinically or thermally.

A “prepolymer” refers to a starting polymer which can be cured (e.g., crosslinked and/or polymerized) actinically or thermally or chemically to obtain a crosslinked and/or polymerized polymer having a molecular weight much higher than the starting polymer. A “crosslinkable prepolymer” refers to a starting polymer which can be crosslinked upon actinic radiation or heating to obtain a crosslinked polymer having a molecular weight much higher than the starting polymer.

The term “soluble”, in reference to a compound or material in a solvent, means that the compound or material can be dissolved in the solvent to give a solution with a concentration of at least about 0.05% by weight at room temperature (i.e., from about 22° C. to about 28° C.).

The term “insoluble”, in reference to a compound or material in a solvent, means that the compound or material can be dissolved in the solvent to give a solution with a concentration of less than 0.005% by weight at room temperature (as defined above).

As used in this application, the term “molecular weight” of a polymeric material (including monomeric or macromeric materials) refers to the number average molecular weight unless otherwise specifically noted or unless testing conditions indicate otherwise.

As used in this application, the term “long-lasting surface hydrophilicity and wettability” in reference to a contact lens means that the contact lens has a water-break-up time (WBUT) of at least 10 seconds after 30 cycles of digital rubbing treatment or after simulated abrasion cycling treatment.

As used in this application, the term “long-lasting lubricity” in reference to a contact lens means that the contact lens exhibits a mean static COF of less than 0.4, or a mean low kinetic COF of less than 0.25, or a mean high kinetic COF of less than 0.4, after 30 cycles of digital rubbing treatment or after simulated abrasion cycling treatment.

As used in this application, the term “30 cycles of digital rubbing treatment” or means that contact lenses are subjected to 30 repetitions of a digital rubbing procedure which essentially consists of digitally rubbing (wearing disposable powder-free latex gloves) contact lenses with RENU® multi-purpose lens care solution (or an equivalent, i.e., a multi-purpose lens care solution disclosed in Table I of U.S. Pat. No. 5,858,937) for 20 seconds and then rinsing the digitally-rubbed contact lenses with a phosphate-buffered saline for at least 20 seconds. The 30 cycles of digital rubbing treatment can reasonably imitate daily cleaning and disinfecting in a 30-days lens care regime.

The present invention also provides a packaging system for the storage of contact lenses. These lenses can provide optical correction, wound care, drug delivery, diagnostic functionality or cosmetic enhancement or effect or a combination of these properties. The invention is applicable to soft, hydrogel contact lenses. As is understood by one skilled in the art, a lens is considered to be “soft” if it can be folded back upon itself without breaking. As used herein, a hydrogel contact lens refers to a polymeric lens that has the ability to absorb and retain water in an equilibrium state. In the context of the present description, a hydrogel lens can be a polymeric material that is free of a silicone-containing component, or a hydrogel lens can be a polymeric material that includes a silicone-containing component.

Many silicone-free hydrogel contact lenses are based on polymerizable lens formulations that include one or more monomers such as diacetone acrylamide (DA); N,N-dimethylacrylamide (DMA); 2-Hydroxyethyl methacrylate (HEMA); methacrylic acid (MAA); methyl methacrylate (MMA); N-carboxl vinyl ester (NCVE); N-vinyl pyrrolidone (NVP); glyceryl methacrylate (GMA); and 2-methacryloyloxyethyl phosphorylcholine (MPC). Some examples of hydrogel contact lens materials include materials having the following US Adopted Names (USANs): etafilcon, hilafilcon A, hilafilcon B, nelfilcon, nesofilcon, ocufilcon, and omafilcon.

Silicone-containing hydrogel contact lenses are frequently referred to as silicone hydrogel contact lenses. Many silicone hydrogel contact lenses are based on polymerizable lens formulations that include siloxane monomers, oligomers, or macromers. Some examples of silicone hydrogel contact lens materials include materials having the following USANs: aquafilcon, balafilcon, comfilcon, enfilcon, galyfilcon, kalafilcon, lenefilcon, lotrafilcon A, lotrafilcon B, samfilcon, and senofilcon.

The present contact lenses may be the polymerized reaction product of a polymerizable composition that comprises one or more hydrophilic monomers, one or more hydrophobic monomers, one or more silicone-containing monomers, oligomers, or macromers, one or more polymers, or combinations thereof in addition, the polymerizable compositions used to make the present lenses may include crosslinking agents, free radical initiators, tinting agents, UV absorbers, and the like. The present soft contact lenses may comprise, consist essentially of, or consist of, any of the foregoing contact lens materials identified by the USAN names above. Most preferably, the present soft contact lenses may comprise, consist essentially of, or consist of hilafilcon B, balafilcon, or kalafilcon.

The present contact lenses can be molded contact lenses, such as spin-cast molded or cast molded contact lenses, or lathed contact lenses. It can be appreciated that these types of contact lenses can have different physical features resulting from their method of manufacture. A cast molded contact lens refers to a contact lens obtained from a contact lens mold assembly formed from two contact lens mold sections in contact with each other to form a contact lens shaped cavity. In addition, a portion of the present contact lenses can be polished or smoothed after forming the contact lens. For example, a contact lens that has been cast molded or lathed, or both, can be polished to reduce transition areas or improve edge shapes to provide greater comfort compared to unpolished lenses.

The present contact lenses can be daily wear lenses or extended wear lenses. As used herein, an extended wear contact lens refers to a contact lens that is approved for wearing on a continuous basis for more than 24 hours. Each contact lens of the lens pair can be a daily disposable contact lens (i.e., a contact lens that is worn on a person's eye only once and then discarded). In comparison, as understood by persons of ordinary skill in the art, a daily wear lens is a lens that is worn on a person's eye, and is then cleaned and is worn on the person's eye for at least one additional time. It can be appreciated that daily disposable contact lenses can be physically different, chemically different, or both compared to daily wear and extended wear contact lenses. For example, formulations used to make daily wear or extended wear contact lenses are different than formulations used to make daily disposable contact lenses due to the economic and commercial factors in making substantially larger volumes of daily disposable contact lenses.

The present contact lenses are placed on a patient's eye such that the posterior surface of the lens faces the corneal epithelium of the eye of the patient.

When the contact lenses are cast molded contact lenses, the forming step comprises cast molding a polymerizable composition into the shape of a contact lens, separating the cast molded contact lens from a contact lens mold member, contacting the separated cast molded contact lens with a liquid, inspecting the separated cast molded contact lens, packaging the separated cast molded contact lens in a contact lens package, and/or sterilizing the contact lens in the package, or any combinations thereof.

The polymerizable composition is dispensed onto the concave surface of the first mold member. The second mold member is placed against the first mold member to form a contact lens mold assembly having a contact lens shaped cavity with the polymerizable composition located therein. The contact lens mold assembly is then exposed to heat or light to polymerize the polymerizable composition and form a polymerized contact lens product. The contact lens mold assembly is demolded by separating the first and second mold members. The polymerized contact lens product remains attached to the first or the second mold member and is then delensed or separated from the mold member. The delensed contact lens is contacted with a liquid, which may be a washing liquid, or it may be a packaging liquid. In some methods, the washing liquid includes one or more agents to help extract unreacted or partially reacted ingredients from the delensed contact lens product. Once the lenses are placed in a packaging liquid, the packages can be sealed, and sterilized.

The method of packaging and storing an ophthalmic lens according to the present invention includes at least packaging the ophthalmic lens immersed in an aqueous contact lens packaging solution.

### Suitable Collagens

Collagens suitable for use in the present invention include collagens derived from natural sources and those obtained from recombinant methods. Collagen suitable for use in this invention are those that exhibit sufficient solubility and stability in the ophthalmic solutions as described herein. Suitable recombinant collagens may be those that correspond to the naturally occurring human collagen. Additionally, modifications may be made to the naturally occurring sequences to improve their suitability for use within this invention. Most preferably, the recombinant collagen is a fragment of collagen that exhibits the desired properties.

As used herein, the term “recombinant collagen” refers to refers to the family of at least 28 distinct naturally occurring collagen types including, but not limited to collagen types I, II, III, IV, V, VI, VII, VIII, IX, X, XI, XII, XIII, XIV, XV, XVI, XVII, XVIII, XIX, and XX, prepared using recombinant techniques. The term collagen includes collagen, collagen fragments, collagen-like proteins, triple helical collagen, alpha chains, monomers, gelatin, trimers and combinations thereof. Recombinant expression of collagen and collagen-like proteins is known in the art (see, e.g., Bell, EP 1232182B1, Bovine collagen and method for producing recombinant gelatin; Olsen, et al., U.S. Pat. No. 6,428,978 and VanHeerde, et al., U.S. Pat. No. 8,188,230, incorporated by reference herein in their entireties). In some embodiments, the collagen described herein can be prepared using bovine Type I collagen. Collagens are characterized by a repeating triplet of amino acids, (Gly-X-Y)ₙ, where X and Y may be any amino acid, preferably proline and 4-hydroxyproline. The term “collagen” is particularly preferably understood to mean a peptide having the repetitive motif (Gly-Pro-Y)ₙ and/or (Gly-X-Hyp)ₘ, where X and Y may be any amino acid. The structure of collagen may consist of three intertwined peptide chains of differing lengths.

The percentages cited in connection with the molecular weight distribution of the collagen peptides in the collagen peptide preparations according to the invention relate to % by weight in relation to all collagen peptides contained in the relevant collagen peptide preparation.

In connection with the present invention, the term “collagen peptide” can be understood to mean a peptide which has an amino acid sequence occurring in collagen as defined above. A “collagen peptide” may also be understood to mean a genetically modified collagen peptide, which was obtained by modifying the amino acid sequence of a naturally occurring collagen peptide,

In connection with the present invention, the term “recombinant DNA” denotes an artificially produced or manipulated DNA molecule, which has been produced in vitro by means of genetic engineering methods. In one embodiment, the recombinant DNA is composed of components from different organisms of origin.

In connection with the present invention, a “recombinant or recombinantly produced collagen peptide” is understood to mean a collagen peptide encoded by recombinant DNA.

One class of suitable recombinant collagen fragments is described in U.S. Pat. No. 11,673,940, incorporated herein in its entirety by reference. “Hydrolyzed collagen peptide preparation” as that term is used herein are the collagen peptides resulting from the hydrolysis of a recombinantly produced collagen peptide having a molecular weight in a range from 8 to 100 kDa, wherein the resulting collagen peptides have an average molecular weight of 1 to 7 kDa and a molecular weight in the range from 0.1 to 13.5 kDa.

Another class of suitable recombinant collagen is described in U.S. Pat. No. 12,134,640, incorporated herein in its entirety by reference. Such suitable collagens comprise a polypeptide comprising or consisting of an amino acid sequence derived from a collagen type VI, or a fragment, variant, fusion or derivative thereof, or a fusion of said fragment, variant of derivative thereof, wherein the polypeptide, fragment, variant, fusion or derivative is capable of killing or attenuating the growth of microorganisms.

It will be appreciated by persons skilled in the art that the collagen type VI may be from a human or non-human source. For example, the collagen type VI may be derived (directly or indirectly) from a non-human mammal, such as an ape (e.g. chimpanzee, bonobo, gorilla, gibbon and orangutan), monkey (e.g. macaque, baboon and colobus), rodent (e.g. mouse, rat) or ungulates (e.g. pig, horse and cow).

Thus, by “collagen type VI” (also “collagen VI”) we include naturally occurring human collagen type VI and homologues thereof, such as bovine collagen type VI. In one preferred embodiment, the polypeptide is derived from human collagen type VI.

By an amino acid sequence “derived from” collagen type VI we include amino acid sequences found within the amino acid sequence of a naturally occurring collagen type VI protein. In particular, we include amino acid sequences that comprise at least five contiguous amino acids from the sequence of a naturally occurring collagen type VI, but exclude the full length protein. For example, in one embodiment the amino acid sequence may contain at least 5 contiguous amino acids from collagen type VI, for example at least 6, 7, 8, 9, 10, 11, 12, 13, 14, 15, 16, 17, 18, 19, 20, 21, 22, 23, 24, 25, 30, 40, or 50 contiguous amino acids from collagen type VI. Thus, the amino acid sequence derived from collagen type VI corresponds to a fragment of collagen type VI.

In a further embodiment, the polypeptide may comprise or consist of an intact VWA domain.

By “VWA domain” we include the type A domains of von Willebrand factor, and domains showing homology to the type A domains of von Willebrand factor, as well as VWA-domain containing regions.

In one embodiment, the polypeptide is derived from the α3 chain of collagen type VI. Thus, the polypeptide may be derived from the α3N or α3C regions. For example, the polypeptide may be derived from the N2, N3 or C1 domain of the α3 chain of collagen type VI.

In an alternative embodiment, the polypeptide is derived from the α4 chain of collagen type VI.

In another alternative embodiment, the polypeptide is derived from the α5 chain of collagen type VI.

In a further alternative embodiment, the polypeptide is derived from the α6 chain of collagen type VI.

In a still further alternative embodiment, the polypeptide is derived from the α2 chain of collagen type VI, for example from the α2N region.

Yet another class of suitable recombinant collagen fragments is described in US Patent Application Publication US2025/0011392 A1, incorporated herein in its entirety by reference. Such collagen fragments can be derived from the amino acid sequences of Col1A1, Col1A2, and Col3A1. Non-limiting examples of such sequences are those described by Accession Nos. P02461.4 (human Col3A1) (www.ncbi.nlm.nih.gov/protein/124056490), NP 001029211.1 (bovine Col1A1) (www.ncbi.nlm.nih.gov/protein/77404252), NP 776945.1 (bovine CollA2) (www.ncbi.nlm.nih.gov/protein/27806257) and NP 001070299.1 (bovine Col3A1) (www.ncbi.nlm.nih.gov/protein/116003881), which are incorporated herein by reference.

Such recombinant collagen fragments can be a fragment of the full amino acid sequence of a native collagen molecule capable of forming tropocollagen (trimeric collagen) or the fragment can be a fragment of a modified collagen molecule or truncated collagen molecule having an amino acid sequence at least 70, 80, 90, 95, 96, 97, 98, or 99% identical or similar to a native collagen amino acid sequence (or to a fibril forming region thereof or to a segment substantially comprising [Gly-X-Y]n).

In some embodiments, the collagen fragment disclosed herein can have a molecular weight from about 40 kDa to about 60 kDa. In some embodiments, the collagen fragment can have a molecular weight of about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, about 55 kDa, about 56 kDa, about 57 kDa, about 58 kDa, about 59 kDa, or about 60 kDa. In a particular embodiment, the collagen fragment can have a molecular weight of about 55 kDa.

In some embodiments, the collagen fragment described herein can have an amino acid sequence according to SEQ ID NO: 1. In some embodiments, the collagen fragment can have at least about 70%, at least about 75%, at least about 80%, about 85%, at least about 87.5%, at least about 90%, at least about 92.5%, at least about 95%, at least about 97.5%, at least about 98%, at least about 99% or 100% sequence identity, or similarity to SEQ ID NO: 1.

The amino acid sequence of SEQ ID NO: 1 is:

MYRNLIIATALTCGAYSAYVPSEPWSTLTPDASLESALKDYSQTFGIAIK
SLDADKIKRDSYDVKSGVAVGGLAGYPGPAGPPGPPGPPGTSGHPGSPGS
PGYQGPPGEPGQAGPSGPPGPPGAIGPSGPAGKDGESGRPGRPGERGLPG
PPGIKGPAGIPGFPGMKGHRGFDGRNGEKGETGAPGLKGENGLPGENGAP
GPMGPRGAPGERGRPGLPGAAGARGNDGARGSDGQPGPPGPPGTAGFPGS
PGAKGEVGPAGSPGSNGAPGQRGEPGPQGHAGAQGPPGPPGINGSPGGKG
EMGPAGIPGAPGLMGARGPPGPAGANGAPGLRGGAGEPGKNGAKGEPGPR
GERGEAGIPGVPGAKGEDGKDGSPGEPGANGLPGAAGERGAPGFRGPAGP
NGIPGEKGPAGERGAPGPAGPRGAAGEPGRDGVPGGPGMRGMPGSPGGPG
SDGKPGPPGSQGESGRPGPPGPSGPRGQPGVMGFPGPKGNDGAPGKNGER
GGPGGPGPQGPPGKNGETGPQGPPGPTGPGGDKGDTGPPGPQGLQGLPGT
GGPPGENGKPGEPGPKGDAGAPGAPGGKGDAGAPGERGPPAIAGIGGEKA
GGFAPYYG

The amino acid sequence of SEQ ID NO: 2 is:

DVKSGVAVGGLAGYPGPAGPPGPPGPPGTSGHPGSPGSPGYQGPPGEPGQ
AGPSGPPGPPGAIGPSGPAGKDGESGRPGRPGERGLPGPPGIKGPAGIPG
FPGMKGHRGFDGRNGEKGETGAPGLKGENGLPGENGAPGPMGPRGAPGER
GRPGLPGAAGARGNDGARGSDGQPGPPGPPGTAGFPGSPGAKGEVGPAGS
PGSNGAPGQRGEPGPQGHAGAQGPPGPPGINGSPGGKGEMGPAGIPGAPG
LMGARGPPGPAGANGAPGLRGGAGEPGKNGAKGEPGPRGERGEAGIPGVP
GAKGEDGKDGSPGEPGANGLPGAAGERGAPGFRGPAGPNGIPGEKGPAGE
RGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPP
GKNGETGPQGPPGPTGPGGDKGDTGPPGPQGLQGLPGTGGPPGENGKPGE
PGPKGDAGAPGAPGGKGDAGAPGERGPPAIAGIGGEKAGGFAPYYG.

In some embodiments, the collagen fragment described herein can have an amino acid chain length from about 350 amino acids to about 600 amino acids and can have at least about 70%, at least about 75%, at least about 80%, at least about 85%, at least about 87.5%, at least about 90%, at least about 92.5%, at least about 95%, at least about 97.5%, at least about 98%, at least about 99% or 100% sequence identity, or similarity to, SEQ ID NO: 1. In some embodiments, the such a collagen fragment described herein can have a length of about 350 amino acids, about 370 amino acids, about 390 amino acids, about 400 amino acids, about 420 amino acids, about 440 amino acids, about 460 amino acids, about 480 amino acids, about 500 amino acids, about 510 amino acids, about 520 amino acids, about 530 amino acids, about 540 amino acids, about 550 amino acids, about 560 amino acids, about 570 amino acids, about 580 amino acids, about 590 amino acids, or about 600 amino acids.

The present invention, in another aspect, provides a soft hydrogel contact lens, comprising a polymer matrix and one or more collagen fragments which are not covalently linked to the polymer matrix. Such collagen fragments may be fully within the lens, but preferably, at least some of the collagen fragments have at least a portion of their structure at the surface of the soft hydrogel lens. Such soft contact lenses are preferably capable of gradually releasing the collagen fragment over an extended period of wearing time.

In some embodiments, the contact lenses of the present invention in their fully hydrated state can comprise from about 0.0005% to about 25% by weight of a recombinant collagen fragment, from about 0.001% to about 20% by weight of a recombinant collagen fragment, from about 0.01% to about 15% by weight of a recombinant collagen fragment, from about 0.1% to about 10% by weight of a recombinant collagen fragment, from about 0.5% to about 5% by weight of a recombinant collagen fragment, from about 0.7% to about 5% by weight of recombinant collagen fragment, from about 1% to about 5% by weight of recombinant collagen fragment, from about 2% to about 5% by weight of recombinant collagen fragment, from about 2% to about 5% by weight of recombinant collagen fragment, from about 3% to about 5% by weight of recombinant collagen fragment, from about 4% to about 5% by weight of recombinant collagen fragment, or about 5% by weight of recombinant collagen fragment.

In some embodiments, the contact lenses of the present invention in their fully hydrated state can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a recombinant collagen fragment. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a collagen fragment with a sequence according to SEQ ID NO: 1 or SEQ ID NO: 2. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24% or about 25% by weight of one or more hydrolysis products of a recombinant collagen fragment. In some embodiments, the composition can comprise about 0.0005%, about 0.001%, about 0.010%, about 0.1%, about 0.2%, about 0.30%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of one or more hydrolysis products of a recombinant collagen fragment with sequences according to one or more of SEQ ID NOs: 1-2. In some embodiments, the composition can comprise about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a mixture of a recombinant collagen fragment and one or more hydrolysis products of that recombinant collagen fragment. In some embodiments, the cosmetic composition can about 0.0005%, about 0.001%, about 0.01%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, by weight of a mixture of a recombinant collagen fragment with a sequence according to SEQ ID NO: 1 or SEQ ID NO: 2 and one or more hydrolysis products of that recombinant collagen fragment.

The method of packaging and storing an ophthalmic lens according to the present invention includes at least packaging the ophthalmic lens immersed in the aqueous contact lens packaging solution described above. The method may include immersing the ophthalmic lens in an aqueous contact lens solution prior to delivery to the customer/wearer, directly following manufacture of the contact lens. Alternately, the packaging and storing in the solution of the present invention may occur at an intermediate point before delivery to the ultimate customer (wearer) but following manufacture and transportation of the lens in a dry state, wherein the dry lens is hydrated by immersing the lens in the contact lens packaging solution. Consequently, a package for delivery to a customer may include a sealed container containing one or more unused contact lenses immersed in an aqueous contact lens packaging solution according to the present invention.

In one embodiment, the steps leading to the present ophthalmic device packaging system include (1) molding an ophthalmic device in a mold comprising at least a first and second mold portion, (2) removing the lens from the mold portions; (3) introducing the packaging solution of this invention and the ophthalmic lens into the container, and (4) sealing the container. Preferably, the method also includes the step of sterilizing the contents of the container. Sterilization may take place prior to, or most conveniently after, sealing of the container and may be effected by any suitable method known in the art, e.g., by balanced autoclaving of the sealed container at temperatures of about 120° C. or higher. Preferred packages are plastic blister packages, including a recess for receiving a contact lens and the packaging solution, where the recess is sealed with lidstock prior to sterilization of the package contents.

### Coefficient of Friction

Tribology is the study of how two surfaces interact with each other when in relative motion. One aspect of tribology that may be of importance to contact lenses is friction. Friction is a measure of a material's resistance to lateral motion when placed against a specific substrate. The relative friction between two surfaces may be described in terms of a coefficient of friction (COF), which is defined as the ratio of the lateral force (Fₓ) that is required to initiate and then sustain movement to the normal force (F_{N}). Further, there are two friction coefficients that may be considered, the peak (or static) and average (or kinetic). The static COF is a measure of how much Fₓ is needed to initiate relative motion of two surfaces and is typically the larger of the two values. Practically, for contact lenses, the static COF is related to the amount of force needed to start a blink cycle or for the lens to begin moving over the cornea. The kinetic COF is a measure of how much lateral force is needed to sustain movement at a particular velocity averaged over a finite period of time. This value is related to the amount of force required to sustain the blink over the course of the entire cycle and the ease of motion of the lens on the cornea (which may be further related to how much the lens moves on the cornea).

Various in vitro techniques are used to determine surface CoF in contact lenses however, an internationally agreed gold standard method does not currently exist. Such tribological testing can be performed, for example, on a TA Discovery Hybrid controlled stress rheometer equipped with a ring tool of ~50 nm surface roughness and designed to be used in an aqueous environment to eliminate effects of moisture loss from the surface. The tool is lowered onto the lens surface until a target normal applied force F_{N} 0.05N is reached. The static, low speed kinetic, and high speed kinetic frictional forces are measured in 3 succinct steps. Each value represents the average of 4-5 lenses. Controls can be run from the same lenses but do not contain recombinant collagen fragments nor subjected to a packaging solution containing recombinant collagen fragments.

To measure the static coefficient of friction the rub tool torque is ramped at 2 μN·m/s from 0 to 500 μN·m while monitoring the rub tool velocity (v). When the torque applied to the rub tool exceeds the static friction force of the lens surface the tool begins to spin freely. The torque at the point the disk begins to spin, the radius of the disk, and the applied normal force are then utilized to calculate the static coefficient of friction.

To measure the Low Speed Kinetic Coefficient of Friction the rub tool is rotated at a constant ‘low’ speed of 0.25 rad/s and the tangential force recorded and averaged for 90 s to calculate the low speed kinetic CoF.

To measure the “High Speed Kinetic Coefficient of Friction” the rub tool rotation is increased to 40.0 rad/s for 30 s and again the tangential force is averaged and used to calculate the high speed kinetic CoF.

A lens containing recombinant collagen fragments preferably exhibits a mean static COF of less than 0.4, or a mean low kinetic COF of less than 0.25, or a mean high kinetic COF of less than 0.4, and in any event less than an identical lens having no collagen fragments. The mean static COF is more preferably less than 0.25, less than 0.2, less than 0.15, or less than 0.1. A lens of the present invention preferably has a mean static COF less than an otherwise identical lens having no collagen fragments. The mean low kinetic COF is preferably less than 0.2, less than 0.15, less than 0.1, or less than 0.05. A lens of the present invention preferably has a mean low kinetic COF less than an otherwise identical lens having no collagen fragments. The mean high kinetic COF is preferably less than 0.35, less than 0.3, less than 0.25, or less than 0.20. A lens of the present invention preferably has a mean high kinetic COF less than an otherwise identical lens having no collagen fragments.

Wettability of lenses can be determined using a captive bubble contact angle technique using a First Ten Angstroms FTA-1000 drop Shape Instrument. All samples were rinsed in 18.2 MΩ deionized HPLC grade water prior to analysis in order to remove components of the packaging solution from the sample surface. Advancing and receding captive bubble contact angles were collected for each sample. The advancing contact angle is defined as the angle measured in water as the air bubble is retracting from the lens surface (water is advancing across the surface). All captive bubble data was collected using a high speed digital camera focused onto the sample/air bubble interface. The contact angle was calculated at the digital frame just prior to contact line movement across the sample/air bubble interface. The receding contact angle is defined as the angle measured in water as the air bubble is expanding across the sample surface (water is receding from the surface).

A lens containing recombinant collagen fragments preferably exhibits a captive bubble contact angle value of less than 80° or in any event less than an identical lens having no collagen fragments. The mean captive bubble contact angle is more preferably less than 80°, less than 70°, less than 60°, less than 50°, or less than 40°. A lens of the present invention preferably has a captive bubble contact angle less than an otherwise identical lens having no collagen fragments.

Wettability of lenses can also be determined using a sessile drop technique measured using KRUSS DSA-100TM instrument at room temperature and using DI water as probe solution. Lenses are blotted dry with a kim wipe to remove surface water without drying the lens, and the lens is mounted on an orb-shaped mount. The cuvette is empty during testing, and the syringe is lowered from above and dispenses the water droplet onto the lens. Measurement is taken in the DSA 100-Drop Shape Analysis Software and recorded.

A lens containing collagen fragments in its fully hydrated state preferably exhibits a sessile water contact angle less than about 80 degrees, preferably less than about 70 degrees, more preferably less than about 60 degrees, most preferably less than about 50 degrees. A lens of the present invention preferably has a sessile water contact angle less than an otherwise identical lens having no collagen fragments.

The surface hydrophilicity of a contact lens (or a biomedical device or a material) is assessed by determining water-break-up time (WBUT), i.e., the time required for the water film to start breaking on the lens surface. Briefly, lenses are removed from the vial and placed in PBS (phosphate buffered saline) for at least two rinses of 30 minutes each and then transferred to fresh PBS in order to remove loosely bound packaging additives from the lens surface. Contact lenses are loaded onto a support pedestal which is submerged in an aqueous solution. A vertical lift stage brings the lens out of the solution, exposing the lens to air which is synchronized to a camera. A video is obtained of the lens dewetting and analyzed on frame-by-frame basis with a post-processing program in Matlab. Lenses exhibiting WBUT>10 seconds are considered to have a hydrophilic surface and are expected to exhibit adequate wettability (ability to support the tear film) on-eye.

A lens containing collagen fragments in its fully hydrated state preferably exhibits a WBUT of at least 10 seconds, preferably at least about 25 seconds, more preferably at least about 50 seconds, most preferably at least about 75 seconds. A lens of the present invention preferably has a WBUT greater than an otherwise identical lens having no collagen fragments.

It is preferable that the addition of collagen fragments to the polymer matrix of a contact lens not result in cloudiness or a significant reduction in spectral transmissivity in the contact lens. Accordingly, it is preferred that the spectral transmissivity of a contact lens of the present invention be greater than 90%, more preferably greater than 94%, even more preferably greater than 95%, and most preferably greater than 96%, when measured by at least one of ISO 18369-3 or ANSI Z80.20.

1. A soft hydrogel contact lens comprising a polymer matrix and one or more collagen fragments which are not covalently linked to the polymer matrix.

2. The soft hydrogel contact lens of claim 1, wherein said collagen fragments have an average molecular weight of 1 to 7 kDa and a molecular weight in the range from 0.1 to 13.5 kDa.

3. (canceled)

4. The soft hydrogel contact lens of claim 1, wherein said collagen fragments are derived from the group of amino acid sequences consisting of collagen type VI, Col1A1, Col1A2, and Col3A1.

5. The soft hydrogel contact lens of claim 4, wherein the collagen fragment is from about 0.001% to about 20% by weight of the fully hydrated lens.

6. The soft hydrogel contact lens of claim 5, wherein said collagen fragments are derived from collagen type VI.

7. The soft hydrogel contact lens of claim 6, wherein said collagen fragments are derived from the group of amino acid sequences consisting of the α2 chain of collagen type VI, the α3 chain of collagen type VI, the α4 chain of collagen type VI, the α5 chain of collagen type VI, and the α5 chain of collagen type VI.

8. The soft hydrogel contact lens of claim 7, wherein said collagen fragments comprise an intact VWA domain.

9. The soft hydrogel contact lens of claim 1, wherein said collagen fragment has the amino acid sequence of SEQ ID NO: 1.

10. (canceled)

11. The soft hydrogel contact lens of claim 1, wherein said collagen fragment has an amino acid sequence with at least about 70% sequence identity to SEQ ID NO: 1.

12. The soft hydrogel contact lens of claim 11, wherein said collagen fragment has a molecular weight from about 40 kDa to about 60 kDa.

13. (canceled)

14. (canceled)

15. (canceled)

16. The soft hydrogel contact lens of claim 1, wherein said collagen fragment has an amino acid sequence with at least about 70% sequence identity to SEQ ID NO: 2.

17. The soft hydrogel contact lens of claim 16, wherein said collagen fragment has an average molecular weight from about 40 kDa to about 60 kDa.

18. (canceled)

19. The soft hydrogel contact lens of claim 18, wherein the collagen fragment is from about 0.001% to about 20% by weight of the fully hydrated lens.

20. An ophthalmic product comprising a sealed package which comprises a packaging solution and a soft hydrogel contact lens, wherein the hydrogel contact lens comprises a polymer matrix and a collagen fragment which is not covalently linked to the polymer matrix but distributed therein.

21. The ophthalmic product of claim 20, wherein said soft hydrogel contact lens has a mean static COF of less than 0.4.

22. (canceled)

23. (canceled)

24. The ophthalmic product of claim 20, wherein said soft hydrogel contact lens has a captive bubble contact angle of less than 80 degrees.

25. The ophthalmic product of claim 20, wherein said soft hydrogel contact lens has a sessile water contact angle less than about 80 degrees.

26. The ophthalmic product of claim 20, wherein said soft hydrogel contact lens has a water break up time of at least 10 seconds.

27.-31. (canceled)

32. A method of treating Keratoconjunctivitis sicca comprising the step of providing an ophthalmic product to a patient in need thereof, wherein said ophthalmic product comprises a sealed package which comprises a packaging solution and a soft hydrogel contact lens, wherein the hydrogel contact lens comprises a polymer matrix and a collagen fragment which is not covalently linked to the polymer matrix but distributed therein.


SEQUENCE LISTING (2 sequences)

SEQ ID NO: 1 - protein, 608 aa, synthetic construct
MYRNLIIATALTCGAYSAYVPSEPWSTLTPDASLESALKDYSQTFGIAIKSLDADKIKRDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG

SEQ ID NO: 2 - protein, 546 aa, synthetic construct
DVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPAIAGIGGEKAGGFAPYYG